(19) United States
(12) Patent Application Publication
(10) Pub. No.: US 2023/0271001 A1
(43) Pub. Date: Aug. 31, 2023

(54) SKIN TREATMENT DEVICE CAPABLE OF AUTOMATICALLY OUTPUTTING HIGH-FREQUENCY ENERGY AND CONTROL METHOD

(71) Applicant: WONTECH Co., Ltd., Daejeon (KR)

(72) Inventors: Jong Won KIM, Seongnam-si (KR); Jung Hyun KIM, Seongnam-si (KR); Young Seok SEO, Sejong-si (KR); Young Sik KIM, Daejeon (KR)

(21) Appl. No.: 18/059,379

(22) Filed: Nov. 28, 2022

(30) Foreign Application Priority Data
Feb. 28, 2022 (KR) 10-2022-0026420

(51) Int. Cl. A61N 1/06; A61N 5/06; A61N 1/32; A61N 1/08

(52) U.S. Cl. CPC A61N 1/06; A61N 5/0616; A61N 1/328; A61N 1/08; A61N 2005/0628

(57) ABSTRACT

A skin treatment device capable of automatically outputting high-frequency energy is proposed. The skin treatment device includes a tip configured to be in contact with skin and transmit high-frequency energy, a handpiece to which the tip may be mounted, and configured to be gripped by a user and to allow the tip to be located on the skin, and a controller configured to control output of the high-frequency energy so that the high-frequency energy may pass through the handpiece and may be transmitted through the tip, and the tip includes a temperature detecting sensor at a front surface thereof to detect temperature of the contacted skin, and the handpiece includes a pressure detecting sensor configured to detect pressure applied to the tip and an acceleration detecting sensor configured to detect acceleration input in response to movement of the handpiece.

### CROSS REFERENCE TO RELATED APPLICATION

The present application claims priority to Korean Patent Application No. 10-2022-0026420, filed on Feb. 28, 2022, the entire contents of which is incorporated herein for all purposes by this reference.

### BACKGROUND OF THE INVENTION

### Field of the Invention

The present disclosure relates to a skin treatment device and a control method thereof. More particularly, the present disclosure relates to a skin treatment device capable of automatically outputting high-frequency energy and a control method thereof, the skin treatment device being capable of automatically transmitting high-frequency energy to the skin without a specific time limit to cause a thermal effect to treat the skin in a minimally invasive manner.

### Description of the Related Art

Recently, a technique for treating the skin by providing energy to the skin using various energy sources to transform the state of skin tissue or improve tissue characteristics has been widely applied. Skin treatment devices using various energy sources such as laser beams, flash lamps, and ultrasonic waves are being developed. Recently, research on skin treatment devices using RF high-frequency energy is being actively carried out.

When high-frequency energy is provided to the skin surface, whenever the direction of the high-frequency current changes, molecules constituting the skin tissue vibrate and rub against each other, and deep heat is generated by rotational motion, twisting motion, or collision motion. The deep heat increases the temperature of the skin tissue to reorganize a collagen layer, thereby improving wrinkles and changing skin elasticity.

Furthermore, blood circulation of the skin tissue is enhanced and promoted, thereby causing effects of preventing skin aging and improving the overall condition of the skin.

At this point, as the related art about the device for the treatment of skin tissue, Korean Patent Application Publication No. 10-2004-0093706 (Publication Date: Nov. 8, 2004) has been proposed.

The conventional proposed art relates to the handpiece for high-frequency treatment including a handpiece housing, an electrode assembly configured to be removably connected to the handpiece housing, and a fluid delivery member mechanically connected to the electrode assembly. Here, in order for electrode assembly to non-invasively treat a lower tissue of a skin surface by high-frequency energy, the high-frequency energy is configured to be connected electrical-capacitively to the skin tissue and mechanically connected to the electrode assembly. Furthermore, the related art proposed the handpiece device including a non-volatile memory configured to store one or more of a duty cycle for controlling the fluid delivery member, a number of times the electrode assembly moves with respect to the skin surface, and a number of areas treated by the electrode assembly.

The related art is characterized by supplying high-frequency energy to the inside of the human body and stimulating the skin to perform treatment and to control the treatment. In the process, in order to obtain more an efficient and stable treatment effect, while the device maintains contact with the skin of the treatment area, it is important to continuously supply the high-frequency energy with constant power and to uniformly apply the high-frequency energy to the skin of the treatment area.

### SUMMARY OF THE INVENTION

The present disclosure is intended to provide a skin treatment device capable of automatically outputting high-frequency energy and a control method thereof, the skin treatment device being configured to automatically transmit high-frequency energy to the skin without a specific time limit to cause a thermal effect to treat the skin in a minimally invasive manner.

In order to achieve the above objective, according to one aspect of the present invention, there is provided a skin treatment device capable of automatically outputting high-frequency energy, the skin treatment device including: a tip configured to be in contact with skin and transmit high-frequency energy; a handpiece to which the tip may be mounted, and configured to be gripped by a user and to allow the tip to be located on the skin; and a controller configured to control output of the high-frequency energy so that the high-frequency energy may pass through the handpiece and may be transmitted through the tip, wherein the tip may include a temperature detecting sensor at a front surface thereof to detect temperature of the contacted skin, and the handpiece may include a pressure detecting sensor configured to detect pressure applied to the tip and an acceleration detecting sensor configured to detect acceleration input in response to movement of the handpiece.

The tip may further include: an electrode removably coupled to one portion of the handpiece, and when contacting with the skin, provided to transmit the high-frequency energy via the front surface.

The skin treatment device may further include: a high-frequency generator configured to generate the high-frequency energy having specific frequency, waveform, and power and to transmit the high-frequency energy to the tip, wherein the controller may be configured to generate a pulse signal to control at least one of frequency, power, and pulse interval of the high-frequency energy generated from the high-frequency generator and to transmit the pulse signal to the high-frequency generator, so that the high-frequency generator may generate the high-frequency energy corresponding to the pulse signal.

The controller may be configured to detect the temperature of the contacted skin via the temperature detecting sensor as the tip is brought into contact with the skin, and to determine whether the detected temperature is less than a preset temperature value or is equal to or higher than the preset temperature value to determine whether or not the tip is in contact with the skin.

The controller may be configured to detect pressure applied to the tip via the pressure detecting sensor as the tip is brought into contact with the skin, and to determine whether the detected pressure is less than a preset pressure value or equal to or higher than the preset pressure to determine whether or not the tip is in contact with the skin.

The controller may be configured to detect acceleration input as the handpiece moves, via the acceleration detecting sensor, and to determine whether the detected acceleration is less than a preset acceleration value or equal to or higher than the preset acceleration value to determine whether or not the handpiece moves.

A control method of a skin treatment device may include: obtaining a control signal to generate high-frequency energy, the control signal being capable of controlling output of high-frequency energy by a controller; determining whether or not the obtained control signal is a first mode; determining whether or not the obtained control signal is a second mode; and performing an individual mode to control high-frequency energy generation of a high-frequency generator by each of a temperature detecting sensor, a pressure detecting sensor, and an acceleration detecting sensor.

In the determining of the existing of the first mode, the first mode may be a mode generating high-frequency energy corresponding to a pulse signal input only during a specific time, via the high-frequency generator, and in the determining of the existing of the second mode, the second mode may be a mode automatically and repeatedly generating a high-frequency signal corresponding to a pulse signal input without a specific time limit, via the high-frequency generator, and the second mode may be continuously maintained when pressure applied to a tip and movement of a handpiece are detected.

The present disclosure is configured to automatically transmit high-frequency energy on the skin without a specific time limit to cause a thermal effect, so that it is possible to treat the skin in a minimally invasive manner.

Furthermore, the present disclosure is configured to induce continuous movement of the handpiece and to prevent high-frequency energy from being transmitted only to a specific skin area, thereby preventing damages to the skin in advance.

### BRIEF DESCRIPTION OF THE DRAWINGS

FIG. 1 is a perspective view showing a skin treatment device capable of automatically outputting high-frequency energy according to an embodiment of the present disclosure.

FIG. 2 is a block diagram showing schematic elements of the skin treatment device capable of automatically outputting high-frequency energy according to the embodiment of the present disclosure.

FIG. 3 is a front view showing a tip the skin treatment device capable of automatically outputting high-frequency energy according to the embodiment of the present disclosure.

FIG. 4 is a perspective view showing a handpiece of the skin treatment device capable of automatically outputting high-frequency energy according to the embodiment of the present disclosure.

FIG. 5 is a side sectional view showing coupling between the tip and the handpiece of the skin treatment device capable of automatically outputting high-frequency energy according to the embodiment of the present disclosure.

FIG. 6 is a flowchart showing a control method of the skin treatment device capable of automatically outputting high-frequency energy according to an embodiment of the present disclosure.

FIG. 7 is a flowchart showing individual mode performance stages according to an embodiment of the present disclosure.

FIG. 8 is a flowchart showing individual mode performance stages according to another embodiment of the present disclosure.

FIG. 9 is a flowchart showing individual mode performance stages according to a further embodiment of the present disclosure.

### DETAILED DESCRIPTION OF THE INVENTION

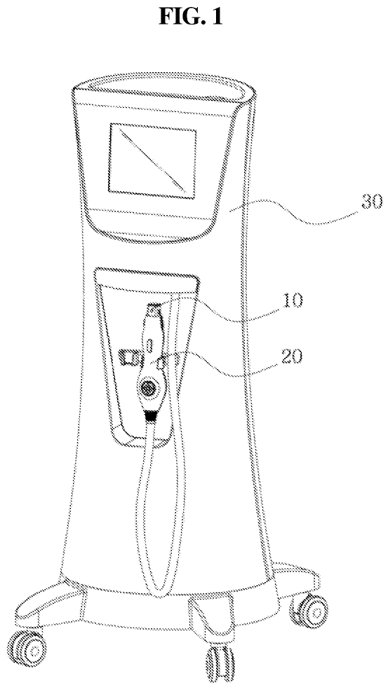

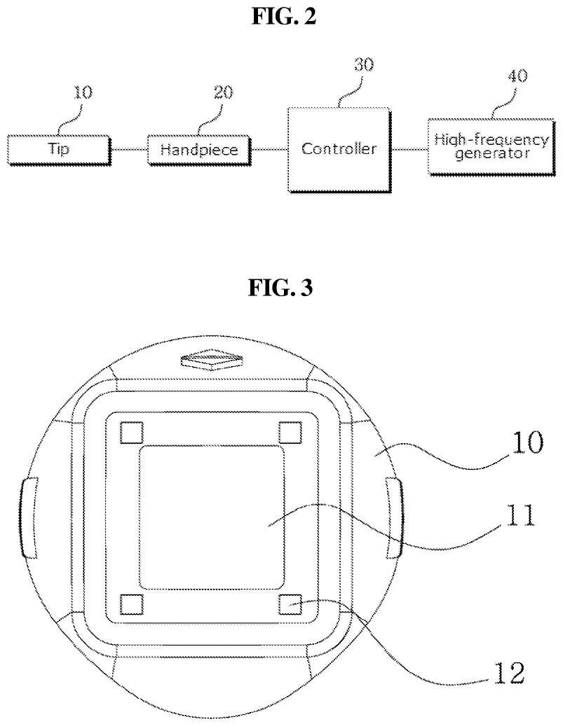

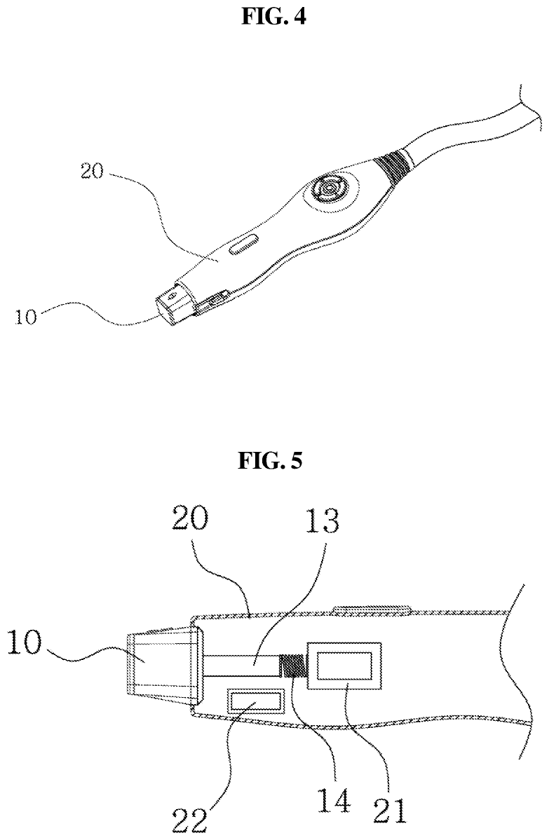

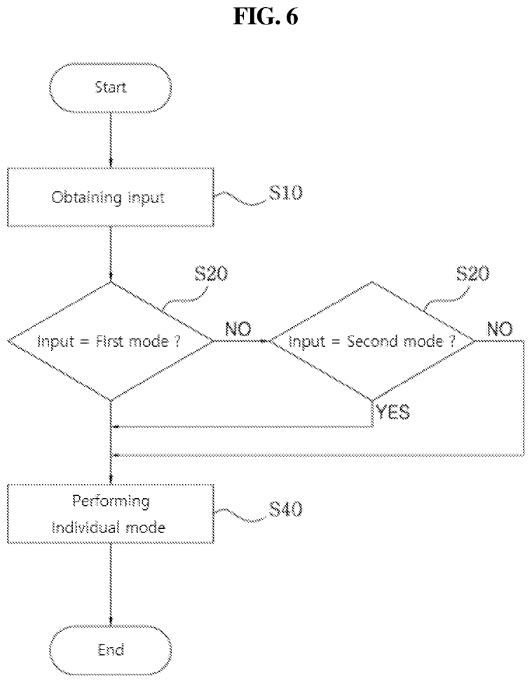

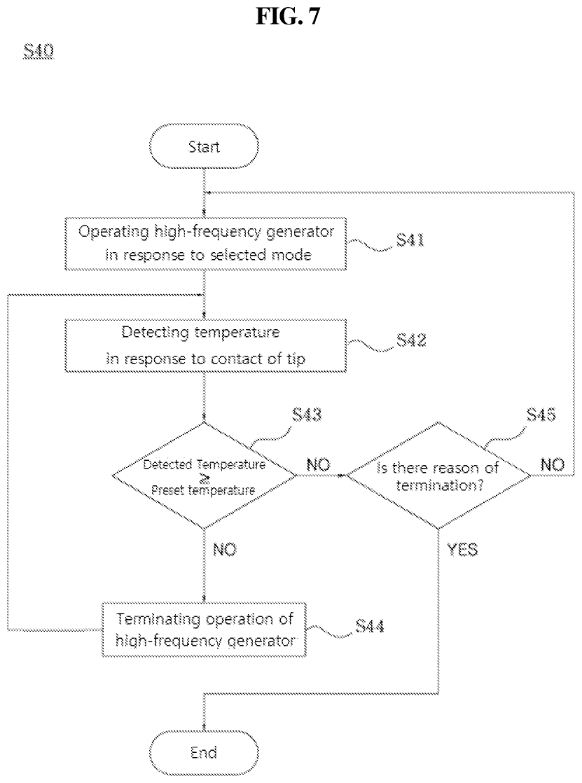

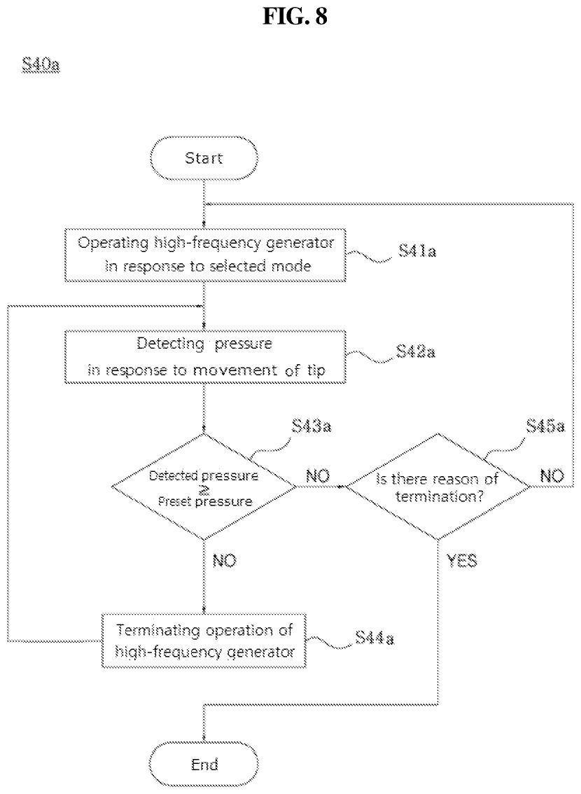

FIG. 1 is a perspective view showing a skin treatment device capable of automatically outputting high-frequency energy according to an embodiment of the present disclosure. FIG. 2 is a block diagram showing schematic elements of the skin treatment device capable of automatically outputting high-frequency energy according to the embodiment of the present disclosure. FIG. 3 is a front view showing a tip the skin treatment device capable of automatically outputting high-frequency energy according to the embodiment of the present disclosure. FIG. 4 is a perspective view showing a handpiece of the skin treatment device capable of automatically outputting high-frequency energy according to the embodiment of the present disclosure. FIG. 5 is a side sectional view showing coupling between the tip 10 and the handpiece 20 of the skin treatment device capable of automatically outputting high-frequency energy according to the embodiment of the present disclosure. FIG. 6 is a flowchart showing a control method of the skin treatment device capable of automatically outputting high-frequency energy according to an embodiment of the present disclosure. FIG. 7 is a flowchart showing individual mode performance stages according to an embodiment of the present disclosure. FIG. 8 is a flowchart showing individual mode performance stages according to another embodiment of the present disclosure.

Hereinbelow, an embodiment of the present disclosure will be described in detail with reference to accompanying drawing.

Referring to FIGS. 1 to 2, the present disclosure may include the tip 10, the handpiece 20, a controller 30, and a high-frequency generator 40 so as to automatically transmit high-frequency energy to skin without a limit of a specific time to generate a thermal effect to treat the skin in a minimally invasive manner.

Referring to FIG. 3, the tip 10 may be brought into contact with the skin to be treated to transmit high-frequency energy to the contacted skin. The tip 10 may provide a uniform thermal effect within the tissue of the skin at a depth selected to minimize or prevent thermal damage to skin surface or tissue.

The tip 10 may be removably attached to the handpiece 20 and receive high-frequency energy from the handpiece 20. The tip 10 includes a rectangular electrode 11 provided on a front surface of the tip 10 and transmitting the high-frequency energy transmitted from the handpiece 20. The electrode 11 may be formed at a portion of the center portion of the front surface of the tip 10. A circumferential surface portion of the electrode 11 may be electrically insulated, and the high-frequency energy may emit via the rectangular electrode 11. A circuit may be provided inside the tip 10 to receive the high-frequency energy from the handpiece 20, and the circuit may be connected to the electrode 11 and transmit the high-frequency energy.

At least one temperature detecting sensor 12 may be formed around the electrode 11. For example, the temperature detecting sensor 12 may be disposed, around the electrode 11, at each corner of the electrode 11. The temperature detecting sensor 12 may detect the temperature and determine whether or not the tip is brought into contact with the electrode in response to the detected temperature. When the electrode 11 is in contact with the skin, the temperature detecting sensor 12 may detect the temperature of the contacted skin. The temperature detecting sensor 12 may detect the temperature in real time while the electrode 11 is in contact with the skin. When the electrode 11 is brought into contact with the skin and the detected skin temperature is equal to or higher than a preset reference temperature, the temperature detecting sensor 12 may determine that the electrode is in contact with the skin.

Referring to FIG. 4, the handpiece 20 may transmit the high-frequency energy to the tip 10. The handpiece 20 may be gripped to allow the tip 10 to be selectively brought into contact with the skin. In order to transmit the high-frequency energy to the tip 10, the handpiece 20 may be connected to the electrode 11 of the tip 10 via a wire, a pin, etc.

More specifically, the handpiece 20 may be formed in a shape for the user to easily grip the handpiece. The handpiece 20 may be formed in an ergonomically flexible shape so that the user can grip the handpiece by a hand. The tip 10 may be removably coupled to a first portion of the handpiece 20 and a second portion thereof may be electrically connected to the controller 30. A button may be provided on one surface of the handpiece 20 to control an on/off state of power of the high-frequency energy or to control an on/off state of transmission of the high-frequency energy. The handpiece 20 may have an adjustment button to adjust output intensity and number of outputs of high-frequency energy. The user can grip the handpiece 20 to manipulate the electrode 11 of the tip 10 so that the electrode 11 is brought into contact with the skin, and transmit the high-frequency energy to the tip 10 by pressing the button.

Here, the handpiece 20 may include a pressure detecting sensor 21 to detect pressure applied to the tip 10 and an acceleration detecting sensor 22 to detect movement of the handpiece 20.

The pressure detecting sensor 21 may control transmission of the high-frequency energy of the tip 10 in response to the pressure applied to the tip 10. The pressure detecting sensor 21 may detect the pressure input in response to movement of the tip 10. The pressure detecting sensor 21 may detect the pressure input in response to movement of the tip 10 and determine whether or not the tip is in contact with the skin, in response to the input pressure. When the tip 10 is in contact with the skin and moves and it is detected that the pressure input by the moving tip 10 is equal to or higher than the preset reference pressure, the pressure detecting sensor 21 may determine that the tip is in contact with the skin.

Referring to FIG. 5, the pressure detecting sensor 21 may be provided inside the handpiece 20 and be mechanically connected to the tip 10. The tip 10 may be selectively brought into contact with the pressure detecting sensor 21 in response to movement thereof.

In the above case, a connection member 13 may be coupled to a rear surface of the tip 10, and an elastic part 14 may be provided between the connection member 13 and the pressure detecting sensor 21. The connection member 13 may be formed in a bar shape having a predetermined length, and a first end of the connection member 13 may be coupled to the rear surface of the tip 10. The elastic part 14 has the elasticity and a first end of the elastic part 14 may be in contact with a second end of the connection member 13, and a second end of the elastic part 14 may be in contact with the pressure detecting sensor 21. As an example, the elastic part 14 may be “a spring”.

Here, when the tip 10 is in contact with the skin, the tip 10 moves toward the inside space of the handpiece 20, and as the connection member 13 moves together with the tip, pressure may be applied to the elastic part 14, and the applied pressure may be transmitted to the pressure detecting sensor 21 through the elastic part 14. After, when the contact between the tip 10 and the skin is released, the tip may be recovered to the original position thereof by an elastic recovery force of the elastic part 14.

Accordingly, the pressure detecting sensor 21 may detect the applied pressure as the tip 10 moves toward the handpiece 20 while protruding on the one portion of the handpiece 20 to control the transmission of the high-frequency energy of the tip 10.

The acceleration detecting sensor 22 may be provided inside the handpiece 20 and may detect acceleration of the handpiece 20. The acceleration detecting sensor 22 may detect the acceleration generated when the handpiece 20 gripped by the user moves or rotates. The acceleration detecting sensor 22 may determine whether or not the handpiece 20 is in a moving state or a stopped state in response to the acceleration of the handpiece 20. When it is detected that the acceleration of the handpiece 20 is equal to or higher than a preset acceleration, the acceleration detecting sensor 22 may determine that movement of the handpiece 20 has been performed.

Therefore, the handpiece 20 is configured to treat the skin by contacting the tip 10 on the skin to be treated, and by transmitting the high-frequency energy received from the controller 30 to the skin, through the button control.

The controller 30 may control output of the high-frequency energy. By controlling the high-frequency generator 40, the output of the high-frequency energy from the high-frequency generator 40 may be controlled. The controller 30 may control the high-frequency generator 40 so that the high-frequency energy having a predetermined frequency, waveform, power, etc. may be output. The controller 30 may control the high-frequency generator 40 so that the high-frequency energy adjusted in a frequency, waveform, power, etc. according to skin properties may be transmitted to the skin through the electrode 11.

The controller 30 may receive a control signal such as power, pulse interval, etc. in order to control the output of the high-frequency energy including the user interface. The controller 30 may be a control device that is configured to control outputting of the high-frequency energy generated from the high-frequency generator 40 to the electrode of the tip 10, according to an input control signal. The controller 30 generates a pulse signal to control the power and the pulse interval of the high-frequency energy and transmit the pulse signal to the high-frequency generator 40, so that generation and outputting of high-frequency energy can be controlled.

Specifically, the controller 30 may control the high-frequency energy generated by the high-frequency generator 40 and transmit the high-frequency energy to the tip 10. The controller 30 may receive a signal from each of the temperature detecting sensor 12 of the tip 10 and the pressure detecting sensor 21 and the acceleration detecting sensor 22 of the handpiece 20 and determine whether or not the tip is in contact with the skin and control whether the transmission of the high-frequency energy through the tip 10. Here, the signal may be the temperature detected by the temperature detecting sensor 12, the pressure detected by the pressure detecting sensor 21, and the acceleration detected by the acceleration detecting sensor 22.

Here, the controller 30 may receive a value of the temperature detected by the temperature detecting sensor 12. The controller 30 may determine whether or not the tip 10 is in contact with the skin in response to the temperature value.

More specifically, when the tip 10 is located to be brought into contact with the skin, and when the electrode 11 is brought into contact with the skin and the temperature detecting sensor 12 detects the temperature and the detected temperature value is transmitted, the controller 30 may determine whether or not the temperature value is less than the preset temperature value or is equal to or higher than the preset temperature value to determine whether or not the tip is in contact with the skin. The controller 30 may transmit a trigger signal to the high-frequency generator 40 in response to whether or not the tip is in contact with the skin on the basis of the temperature value, so that the high-frequency generator 40 may generate and output the high-frequency energy.

Accordingly, when the temperature value transmitted from the temperature detecting sensor 12 is less than the preset temperature value, the controller 30 may determine that the tip 10 is not in contact with the skin and may prevent the high-frequency energy from being transmitted. On the other hand, when the temperature value transmitted from the temperature detecting sensor 12 is equal to or higher than the preset temperature value, the controller 30 may determine that contact between the tip 10 and the skin has been performed, and control the transmission of the high-frequency energy so that the high-frequency energy is safely transmitted to the skin.

Furthermore, the controller 30 may receive a value of the pressure detected by the pressure detecting sensor 21. The controller 30 may determine whether or not the tip 10 is in contact with the skin, in response to the pressure value.

More specifically, when the tip 10 is brought into contact with the skin and the pressure detecting sensor 21 of the handpiece 20 detects pressure and the detected pressure value is transmitted to the controller 30, the controller 30 may determine whether the pressure value is less than or equal to or higher than the preset pressure value to determine whether or not the tip is in contact with the skin. The controller 30 may transmit a trigger signal to the high-frequency generator 40 in response to whether or not the tip is in contact with the skin on the basis of the pressure value, so that the high-frequency generator 40 may generate and output the high-frequency energy.

Accordingly, when the temperature transmitted from the pressure detecting sensor 21 is less than the preset temperature value, the controller 30 may determine that contact between the tip 10 and the skin is not performed and may prevent the high-frequency energy from being transmitted. On the other hand, when the pressure value transmitted from the pressure detecting sensor 21 is equal to or higher than the preset pressure value, the controller 30 may determine that contact between the tip 10 and the skin is performed, and control the transmission of the high-frequency energy so that the high-frequency energy is safely transmitted to the skin. When a pressure value equal to or higher than the preset pressure value is continuously detected, the controller 30 may maintain the transmission of the high-frequency energy transmitting to the tip 10.

Furthermore, the controller 30 may receive a value of the acceleration detected by the acceleration detecting sensor 22. The controller 30 may determine movement of the handpiece 20 in response to the acceleration value.

More specifically, when the tip 10 is brought into contact with the skin and the acceleration detecting sensor 22 of the handpiece 20 detects acceleration and the detected acceleration value is transmitted to the controller, the controller 30 may determine whether the acceleration value is less than or equal to or higher than the preset acceleration value to determine movement of the handpiece 20 such that whether or not the handpiece 20 moves by a predetermined distance on the skin. The controller 30 may transmit a trigger signal to the high-frequency generator 40 in response to whether or not the tip moves on the skin on the basis of the acceleration value, so that the high-frequency generator 40 may generate and output the high-frequency energy.

Accordingly, when the acceleration value transmitted from the acceleration detecting sensor 22 less than the preset acceleration value, the controller 30 may determine that the movement of the handpiece 20 on the skin is not performed and prevent the transmission of the high-frequency energy. On the other hand, when the acceleration value transmitted from the acceleration detecting sensor 22 is equal to or higher than the preset acceleration value, the controller 30 may determine that movement of the handpiece 20 on the skin is performed and control the transmission of the high-frequency energy so that the high-frequency energy is safely transmitted to the skin. When an acceleration value equal to or higher than the preset value is continuously detected, the controller 30 may maintain the transmission of the high-frequency energy transmitting to the tip 10.

Accordingly, the controller 30 may receive the temperature, pressure, and acceleration respectively detected by the sensors provided in the tip 10 and the handpiece 20 and determine contact between the tip 10 and the skin and movement of the handpiece 20 on the basis of the temperature, pressure, and acceleration, thereby controlling the high-frequency energy of the tip 10.

For example, when contact between the tip 10 and the skin is performed on the basis of temperature and pressure while the tip 10 is in contact with the skin, the controller 30 may transmit the high-frequency energy to the tip 10, and then when movement of the handpiece 20 is performed on the basis of acceleration, the controller may continuously transmit the high-frequency energy to transmit more high-frequency energy to a specific region of the skin, so that skin burns can be prevented in advance.

In other words, when the electrode 11 is in contact with the skin or moves on the skin during the treatment, the controller 30 performs the control so that the high-frequency energy is automatically transmitted to the skin.

The high-frequency generator 40 may generate the high-frequency energy. The high-frequency generator 40 may be provided inside a closure-shaped housing (not shown) together with the controller 30. The high-frequency energy generated by the high-frequency generator 40 may be “radio frequency (RF)”. The high-frequency generator 40 may generate high-frequency energy into specific frequency, waveform, and power. The high-frequency generator 40 may generate and output high-frequency energy having various waveforms such as square wave, triangular wave and sine wave. The high-frequency generator 40 may transmit the generated high-frequency energy to the tip 10. Here, the high-frequency generator 40 may be controlled by the controller 30 to generate the high-frequency energy into specific frequency, waveform, and power. The energy output from the high-frequency generator 40 may be transmitted to the electrode 11 of the tip 10 through the handpiece 20.

Hereinbelow, according to an embodiment of the present disclosure, a control method of the skin treatment device capable of automatically outputting high-frequency energy will be described in detail. Here, description overlapped with the description of the high-frequency energy transmission device according to the embodiment of the present disclosure mentioned above will be omitted or simplified.

FIG. 6 is a flowchart showing a control method of the skin treatment device capable of automatically outputting high-frequency energy according to an embodiment of the present disclosure.

Referring to FIG. 6, the control method may include obtaining, at S10, the control signal, wherein the skin treatment device capable of automatically outputting high-frequency energy obtains the control signal capable of controlling the output of high-frequency energy via the controller 30 so as to generate the high-frequency energy.

The control method of the skin treatment device capable of automatically outputting high-frequency energy may include determining the obtaining input at S20, such that whether the obtaining input is a first mode or a second mode. When the obtained input is not the first mode or the second mode, the controller 30 may terminate the operation of the skin treatment device capable of automatically outputting high-frequency energy.

Here, the first mode may be a mode to generate high-frequency energy corresponding to a pulse signal input only during a specific time, through the high-frequency generator 40. The second mode may be a mode to automatically and repeatedly generate a high-frequency signal corresponding to a pulse signal input without a specific time limit, through the high-frequency generator 40. The second mode may be continuously maintained when the movement of the handpiece 20 is detected in addition to the pressure applied to the tip 10.

The control method of the skin treatment device capable of automatically outputting high-frequency energy may include performing, at S40, an individual mode. In the performing, at S40, of the individual mode, the controller 30 may control the high-frequency generator 40 and perform an individual mode via each of the temperature detecting sensor 12, the pressure detecting sensor 21, and the acceleration detecting sensor 22.

FIG. is a flowchart showing the performing at S40 of an individual mode according to an embodiment of the present disclosure.

Referring to FIG. 7, the performing at S40 of an individual mode may include operating, at S41, the high-frequency generator in response to the selected mode. Here, the operating at S41 of the high-frequency generator may be performed by the controller 30.

The performing at S40 of an individual mode may include detecting, at S42, temperature. In the detecting at S42 of the temperature, the temperature detecting sensor 12 of the tip 10 may obtain temperature information detected as the electrode 11 is contact with the skin.

The performing at S40 of an individual mode may include determining, at S43, the detected temperature. In the determining at S43 of the temperature, the temperature detecting sensor 12 may determine whether or not the electrode 11 is in contact with the skin, and whether or not the temperature of the contacted skin is equal to or higher than the preset reference temperature. The preset reference temperature may mean an average temperature of the human when a contacted state between the skin to which the high-frequency energy is transmitted and the electrode 11 is maintained.

The performing at S40 of an individual mode may include terminating, at S45, the operation of the high-frequency generator. In the terminating at S45 of the operation of the high-frequency generator, the controller 30 may terminate the operation of the high-frequency generator 40. When a detected temperature is less than the preset reference temperature, the controller 30 may perform terminating, at S44, the operation of the high-frequency generator. After the terminating at S44 of the operation of the high-frequency generator, the controller 30 may perform detecting, at S42, the temperature.

The performing at S40 of an individual mode may include determining, at S45, whether or not a terminating reason occurs. The determining at S45 of the occurrence of the terminating reason may be performed by the controller 30. When it is determined that the terminating reason of the performing at S40 of an individual mode occurs, the controller 30 may terminate the performing at S40 of an individual mode. The terminating reason of the performing at S40 of an individual mode may be, for example, a situation of sharply increasing of detected temperature, a situation after it is determined that a temperature detected through the determining of the temperature is equal to or higher than the preset reference temperature, and when the detected temperature is lowered and a temperature detected after a predetermined time is not equal to or higher than the preset reference temperature, or a situation where a termination input is obtained. When it is determined that a terminating reason of the performing at S40 of an individual mode does not occur, operating, at S41, the high-frequency generator in response to the selected mode may be performed.

FIG. 8 is a flowchart showing individual mode performance stages according to another embodiment of the present disclosure.

Referring to FIG. 8, the performing at S40a of an individual mode may include operating, at S41a, the high-frequency generator in response to the selected mode. Here, the operating at S41a of the high-frequency generator may be performed by the controller 30.

The performing at S40a of an individual mode may include detecting, at S42a, pressure. The detecting at S42a of the pressure may be performed by obtaining pressure information detected as the electrode 11 is in contact with the skin or moves on the skin, by the pressure detecting sensor 21.

The performing at S40a of an individual mode may include determining, at S43a, the detected pressure. In the determining at S43a of the detected pressure, the pressure detecting sensor 21 detects pressure input as the electrode 11 is in contact with the skin or moves on the skin, and it may be determined whether or not the detected pressure is equal to or higher than the preset reference pressure. The preset reference pressure may mean a pressure enough to maintain a contacted state between the skin to which the high-frequency energy is transmitted and the electrode 11.

The performing at S40 of an individual mode may include terminating, at S44a, the operation of the high-frequency generator. In the terminating at S44a of the operation of the high-frequency generator, the controller 30 may terminate the operation of the high-frequency generator 40. When a detected pressure is less than the preset reference pressure, the controller 30 may perform terminating, at S44a, the operation of the high-frequency generator. After the terminating at S44a of the operation of the high-frequency generator, the controller 30 may perform detecting, at S42a, the pressure.

The performing at S40a of an individual mode may include determining, at S45a, whether or not the terminating reason occurs. The determining at S45a of the occurrence of the terminating reason may be performed by the controller 30. When it is determined that the terminating reason of the performing at S40a of an individual mode occurs, the controller 30 may terminate the performing at S40a of an individual mode. The terminating reason of the performing at S40 of an individual mode may be, for example, a situation of sharply increasing of input pressure, a situation after it is determined that a pressure detected through the determining at S43a of the pressure is equal to or higher than the preset reference pressure, and when the detected pressure is lowered and a pressure detected after a predetermined time is not equal to or higher than the preset reference pressure, or a situation where a termination input is obtained. When it is determined that a terminating reason of the performing at S40 of an individual mode does not occur, operating, at S41, the high-frequency generator in response to the selected mode may be performed.

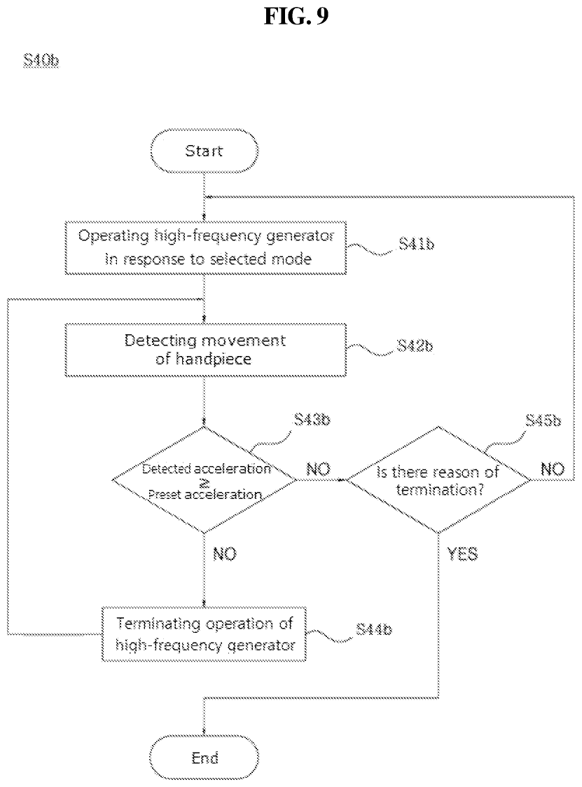

FIG. 9 is a flowchart showing individual mode performance stages according to a further embodiment of the present disclosure.

Referring to FIG. 9, the performing at S40b of an individual mode may include operating, at S41b, the high-frequency generator in response to the selected mode. Here, the operating at S41b of the high-frequency generator may be performed by the controller 30.

The performing at S40b of an individual mode may include detecting, at S42b, the movement of the handpiece 20. The detecting at S42b of the movement of the handpiece 20 may be performed by obtaining the acceleration information detected as the electrode 11 is in contact with the skin or moves on the skin, by the acceleration detecting sensor 22.

The performing at S40b of an individual mode may include determining, at S43b, the detected acceleration. In the detecting at S43 of the acceleration, the acceleration detecting sensor 22 may detect the acceleration input by contact with the skin or movement of the electrode, and may determine whether or not the detected acceleration is equal to or higher than the preset reference acceleration. The preset reference acceleration may mean acceleration enough for the handpiece 20 to move by the predetermined distance while the tip 10 is in contact with the skin.

The performing at S40b of an individual mode may include terminating, at S44b, the operation of the high-frequency generator. In the terminating at S44b of the operation of the high-frequency generator, the controller 30 may terminate the operation of the high-frequency generator 40. When a detected acceleration is less than the preset reference acceleration, the controller 30 may perform terminating, at S44b, the operation of the high-frequency generator. After the terminating at S44b of the operation of the high-frequency generator, the controller 30 may perform detecting, at S42b, the temperature.

The performing at S40b of an individual mode may include determining, at S45b, whether or not a terminating reason occurs. The determining at S45b of the occurrence of the terminating reason may be performed by the controller 30. When it is determined that the terminating reason of the performing at S40b of an individual mode occurs, the controller 30 may terminate the performing at S40b of an individual mode. The terminating reason of the performing at S40 of an individual mode may be, for example, a situation where a detected acceleration is not equal to or higher than the preset reference acceleration, or a situation where termination input is obtained. When it is determined that a terminating reason of the performing at S40b of an individual mode does not occur, operating, at S41b, the high-frequency generator in response to the selected mode may be performed.

Therefore, according to the present disclosure, the skin treatment device capable of automatically outputting high-frequency energy may stably transmit the high-frequency energy while the tip 10 continuously maintains contact with the skin, and it is possible to prevent the contact between the skin and the tip from being separated from each other.

Specifically, according to the present disclosure, when the high-frequency energy is supplied through the second mode, in repeatedly outputting the high-frequency energy a number of times into the same frequency, waveform, power, etc., after supply of the high-frequency energy corresponding to the pulse signal is performed, in the automation process where energy is repeatedly supplied, pressure is applied to the tip 10 constantly or a specific pressure or more is applied to the tip 10, and it is determined whether or not the movement of the handpiece 20 is performed, so that the stable high-frequency energy can be transmitted in addition to the reduction of the treatment time.

What is claimed is:

1. A skin treatment device capable of automatically outputting high-frequency energy, the skin treatment device comprising:
a tip (10) configured to be in contact with skin and transmit high-frequency energy;
a handpiece (20) to which the tip (10) is mounted, and configured to be gripped by a user and to allow the tip (10) to be located on the skin; and
a controller (30) configured to control output of the high-frequency energy so that the high-frequency energy passes through the handpiece (20) and is transmitted through the tip (10),
wherein the tip (10) comprises a temperature detecting sensor (12) at a front surface thereof to detect temperature of the contacted skin, and the handpiece (20) comprises a pressure detecting sensor (21) configured to detect pressure applied to the tip (10) and an acceleration detecting sensor (22) configured to detect acceleration input in response to movement of the handpiece (20).

2. The skin treatment device of claim 1, wherein the tip (10) further comprises an electrode (11) removably coupled to one portion of the handpiece (20), and when contacting with the skin, provided to transmit the high-frequency energy via the front surface.

3. The skin treatment device of claim 2, further comprising:
a high-frequency generator (40) configured to generate the high-frequency energy having specific frequency, waveform, and power and to transmit the high-frequency energy to the tip (10),
wherein the controller (30) is configured to generate a pulse signal to control at least one of frequency, power, and pulse interval of the high-frequency energy generated from the high-frequency generator (40) and to transmit the pulse signal to the high-frequency generator (40), so that the high-frequency generator generates the high-frequency energy corresponding to the pulse signal.

4. The skin treatment device of claim 1, wherein the controller (30) is configured to detect the temperature of the contacted skin via the temperature detecting sensor (12) as the tip (10) is brought into contact with the skin, and to determine whether the detected temperature is less than a preset temperature value or is equal to or higher than the preset temperature value to determine whether or not the tip is in contact with the skin.

5. The skin treatment device of claim 1, wherein the controller (30) is configured to detect pressure applied to the tip (10) via the pressure detecting sensor (21) as the tip (10) is brought into contact with the skin, and to determine whether the detected pressure is less than a preset pressure value or equal to or higher than the preset pressure to determine whether or not the tip is in contact with the skin.

6. The skin treatment device of claim 1, wherein the controller (30) is configured to detect acceleration input as the handpiece (20) moves, via the acceleration detecting sensor (22), and to determine whether the detected acceleration is less than a preset acceleration value or equal to or higher than the preset acceleration value to determine whether or not the handpiece (20) moves.

7. A control method of a skin treatment device, the control method comprising:
obtaining (S10) a control signal to generate high-frequency energy, the control signal being capable of controlling output of the high-frequency energy by a controller (30);
determining (S20) whether or not the obtained control signal is a first mode;
determining (S30) whether or not the obtained control signal is a second mode; and
performing (S40) an individual mode to control high-frequency energy generation of a high-frequency generator (40) by each of a temperature detecting sensor (12), a pressure detecting sensor (21), and an acceleration detecting sensor (22).

8. The control method of claim 7, wherein in the determining (S20) of the existing of the first mode, the first mode is a mode generating high-frequency energy corresponding to a pulse signal input only during a specific time, via the high-frequency generator (40), and
in the determining (S30) of the existing of the second mode, the second mode is a mode automatically and repeatedly generating a high-frequency signal corresponding to a pulse signal input without a specific time limit, via the high-frequency generator (40), and the second mode is continuously maintained when pressure applied to a tip (10) and movement of a handpiece (20) are detected.
